(19) United States
(12) Patent Application Publication
(10) Pub. No.: US 2006/0094787 A1
(43) Pub. Date: May 4, 2006

(54) Pharmaceutical composition comprising 5-methyl-2-2'(chloro-6'-fluoroanilino) phenylacetic acid

(76) Inventors: Patrick Forenzo, Glen Gardner, NJ (US); Maha Y. Khaled, Livingston, NJ (US); Barbara Wang, Berkeley Heights, NJ (US); Joseph Lawrence Zielinski, Florham Park, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/549,249

(22) PCT Filed: Mar. 11, 2004
(86) PCT No.: PCT/EP04/02528

§ 371 Date: Nov. 10, 2005

Related U.S. Application Data
(60) Provisional application No. 60/454,145, filed on Mar. 12, 2003.

(51) Int. Cl. A61K 31/195; A61K 9/00

(52) U.S. Cl. 514/567; 424/400

(57) ABSTRACT

Provided are compositions comprising aqueous suspensions of 5-methyl-2-(2′-chloro-6′-fluoroanilino)phenylacetic acid suitable for oral administration. Methods for making such compositions and methods for their stabilization are provided.

This invention relates to compositions for the treatment of cyclooxygenase-2 mediated disorders and conditions comprising 5-methyl-2-(2′-chloro-6′-fluoroanilino)phenylacetic acid or a pharmaceutically acceptable salt thereof suitable for oral administration, and methods of treatment of cyclooxygenase-2 mediated disorders and conditions by the oral administration of 5-methyl-2-(2′-chloro-6′-fluoroanilino)phenylacetic acid.

All patents, patent applications, and other publications referred to herein are hereby expressly incorporated by reference in their entirety. In case of a conflict between the present specification and material incorporated by reference, the present specification is controlling.

The present invention is directed to a composition for the treatment of cyclooxygenase-2 mediated disorders and conditions, the composition comprising a suspension of 5-methyl-2-(2′-chloro-6′-fluoroanilino)phenylacetic acid. The utility of this compound and methods for its synthesis are disclosed in U.S. Pat. No. 6,291,523.

The present invention is also directed to methods for treating a cyclooxygenase-2 dependent disorder or condition comprising administering an effective amount of the compositions of the invention, i.e., a liquid oral dosage formulation comprising of 5-methyl-2-(2′-chloro-6′-fluoroanilino)phenylacetic acid.

As discussed in U.S. Pat. No. 6,291,523, a genus of compounds, including 5-methyl-2-(2′-chloro-6′-fluoroanilino)phenylacetic acid, is useful for the relief of pain, fever and inflammation of a variety of conditions including rheumatic fever, symptoms associated with influenza or other viral infections, common cold, low back and neck pain, dysmenorrhea, headache, including migraine headache, toothache, sprains and strains, myositis, neuralgia, synovitis, arthritis, including osteoarthritis and rheumatoid arthritis, degenerative joint diseases, gout and ankylosing spondylitis, bursitis, burns, and injuries following surgical and dental procedures. Some individuals, especially children, have difficulty swallowing solid oral dosage formulations. Thus, it is desirable to provide liquid oral dosage formulations comprising 5-methyl-2-(2 ′-chloro-6′-fluoroanilino)phenylacetic acid for the treatment of the aforementioned conditions in individuals who have difficulty swallowing solid oral dosage formulations.

It has now surprisingly been found that a shelf-stable liquid oral dosage formulation comprising 5-methyl-2-(2′-chloro-6′-fluoroanilino)phenylacetic acid can be prepared. The 5-methyl-2-(2′-chloro-6′-fluoroanilino)phenylacetic acid drug substance is relatively water insoluble and also degrades in water, and so the ability to produce a shelf-stable formulation was unexpected. Further, it was surprisingly discovered that the suspendability of the 5-methyl-2-(2′-chloro-6′-fluoroanilino) phenylacetic acid drug substance can be highly dependent on the order of addition of the suspension components, in particular the suspending agent and the buffer.

The liquid oral dosage formulations comprising 5-methyl-2-(2′-chloro-6′-fluoroanilino)phenylacetic acid are preferably suspensions of 5-methyl-2-(2′-chloro-6′-fluoroanilino)phenylacetic acid. Suitable suspending agents include microcrystalline cellulose, carboxymethylcellulose sodium, guar gum, xanthan gum, gellan gum, carrageenan, sodium starch glycolate, and mixtures thereof. Concentrations of suspending agent in the formulations of the invention can range between about 0.1% to about 3%, or between about 0.5% and about 2.5%, or between about 1% and about 2%, or about 1.5%.

The formulations of the invention can also contain a wetting agent, e.g., polysorbate 80, poloxamers, including poloxamer 188, polyethoxylated castor oil and polyethoxylated hydrogenated castor oil, and polyoxyl 40 stearate. Poloxamer 188 has the structure HO(CH₂CH₂O)ₐ(CH(CH₃)CH₂OH)_{b}(CH₂CH₂O)_{c}H, where a is 75, b is 30, and c is 75, with an average molecular weight of about 8350. The wetting agent is present in amounts typically between about 0.1% and about 5%, or between about 0.18% and about 1%, or between about 0.18 and about 0.25%, or between about 0.18 and about 0.22%, or about 0.2%.

The pH of the formulation can range between about 4.3 and 5.5, preferably between about 4.5 and about 5.5 or between about 4.75 and about 5.25. The pH can also range between about 4.9 and about 5.0. Suitable buffers include, e.g., alkaline metal citrate buffers, such as alkaline metal citrate salts with citric acid, alkaline metal acetate buffers, such as sodium acetate salts with acetic acid, and alkaline metal succinate buffers, such as sodium succinate salts with succinic acid, and mixtures thereof.

The formulations typically contain an antifoaming agent, e.g., simethicone, typically added as an emulsion, e.g., a 30% emulsion. Such a 30% emulsion can be added at a concentration of about 0.1% to about 0.25% in the final formulation. Sweeteners such as saccharin, sodium saccharin, aspartame, sucralose, acesulfame potassium, glucose, fructose, lactitol, maltitol, maltose, sorbitol, sucrose, and xylitol can be used. Flavoring agents can also be added to improve compliance.

Suitable preservatives for oral suspensions are known to those of skill in the art and include, e.g., benzoic acid, sorbic acid, parabens (butyl, ethyl, methyl, propyl), sodium benzoate, and sodium propionate. A preservative such as those set forth above, or a mixture thereof, can be present in amounts between about 0.01% and about 0.3%; or between about 0.02% and 0.25%; or between about 0.1% and about 0.2%. In one embodiment, the formulation comprises about 0.02% propyl paraben and about 0.18% methyl paraben. Other embodiments include formulations comprising 0.03% propyl paraben and 0.12% methyl paraben, 0.148% methylparaben and 0.016% propylparaben and formulations comprising 0.1% methyl paraben and 0.1% sorbic acid.

The suspensions of the invention can be made in conventional liquid formulation equipment. In one embodiment, the suspension of the invention is produced by a process comprising admixing water, drug substance, and suspending agent, followed by the addition and admixture of buffer components. Alternatively the suspension of the invention may be prepared by admixing water, suspending agent and buffer system components, followed by the addition and admixture of the drug substance. It has surprisingly been discovered that a suspension cannot be achieved if the buffer components are admixed with drug substance prior to the addition of suspending agent, when the suspending agent is a mixture of microcrystalline cellulose and sodium carboxymethylcellulose.

A pH of between about 4.3 and 5.5 provides a suspension with the most stable drug substance. Formulations with a pH below 4.3 have increased level of a cyclic degradation product, while those above pH 5.5 have increased levels of an oxidative degradation product. Further, increasing the pH of suspension formulations of 5-methyl-2-(2′-chloro-6′-fluoroanilino)phenylacetic acid above about pH 5.5 results in an undesirable increased solubilization of the drug substance.

### EXAMPLES

### Example 1

### Formulation

TABLE 1

| Ingredient | Amount (mg/ml) |
|---|---|
| 5-methyl-2-(2′-chloro-6′-fluoroanilino) | 10.0 |
| phenylacetic acid | |
| Propylparaben | 1.0 |
| Poloxamer 188 | 2.00 |
| Sorbic Acid | 1.0 |
| Simethicone emulsion 30% | 1.00 |
| Flavor | 4.0 |
| Suspending agent: Avicel ® RC591 | 18.00 |
| Propylene glycol | 25.00 |
| Sorbitol solution 70% | 200.00 |
| Citric acid anhydrous | 0.71 |
| Sodium citrate dehydrate | 1.88 |
| Sodium saccharin | 0.50 |
| Water purified, USP | q.s. to 1 ml |

Poloxamer 188 is dissolved in water, followed by dispersion of simethicone and drug substance. Separately, methyl and propylparabens are dissolved in propylene glycol to form a preservative solution. Citric acid, sodium citrate, and sodium saccharin are separately dissolved in water. Avicel® RC591 is then dispersed into the poloxamer 188/simethicone/drug substance mixture and homogenized. The preservative solution is then admixed and homogenized, followed by the sorbitol solution, buffer solution, and flavor. Alternately, the poloxamer 188 is dissolved in water, followed by dispersion of drug substance. Separately, methyl and propylparabens are dissolved in propylene glycol to form a preservative solution. Citric acid, sodium citrate, simethicone and sodium saccharin are separately dissolved/dispersed in water. Avicel® RC591 is then dispersed into the sorbitol solution and homogenized. The preservative solution is then admixed, followed by the sorbitol solution, buffer solution, and flavor. The poloxamer 188/drug susbstance dispersion is then admixed to form the final suspension.

Other formulations can be prepared as indicated above, substituting the other surfactants for poloxamer 188, with the following ingredients:

TABLE 2

| Ingredient | Amount (mg/ml) |
|---|---|
| 5-methyl-2-(2′-chloro-6′-fluoroanilino) | 15.0 |
| phenylacetic acid | |
| Propylparaben | 0.20 |
| Poloxamer 188 | 2.00 |
| Methylparaben | 1.80 |
| Simethicone emulsion 30% | 1.00 |
| Flavor | 5.0 |
| Suspending agent: Avicel ® RC591 | 15.00 |
| Propylene glycol | 25.00 |
| Sorbitol solution 70% | 250.00 |
| Citric acid anhydrous | 0.71 |
| Sodium citrate dihydrate | 1.88 |
| Sodium saccharin | 0.50 |
| Water purified, USP | q.s. to 1 ml |

TABLE 3

| Ingredient | Amount mg/ml |
|---|---|
| 5-methyl-2-(2′-chloro-6′-fluoroanilino) | 12.5 |
| phenylacetic acid | |
| Propylparaben | 0.20 |
| Polysorbate 80 | 2.00 |
| Methylparaben | 1.80 |
| Simethicone emulsion 30% | 1.00 |
| Flavor | 5.0 |
| Suspending agent: Avicel ® RC591 | 15.00 |
| Propylene glycol | 25.00 |
| Sorbitol solution 70% | 250.00 |
| Citric acid anhydrous | 0.71 |
| Sodium citrate dehydrate | 1.88 |
| Ascorbic acid | 10 |
| Sodium saccharin | 0.50 |
| Water purified, USP | q.s. to 1 ml |

TABLE 4

| Ingredient | Amount mg/ml |
|---|---|
| 5-methyl-2-(2′-chloro-6′-fluoroanilino) | 15.0 |
| phenylacetic acid | |
| Propylparaben | 0.20 |
| Methylparaben | 1.80 |
| Simethicone emulsion 30% | 1.00 |
| Flavor | 5.0 |
| Suspending agent: Avicel ® RC591 | 18.00 |
| Propylene glycol | 25.00 |
| Sorbitol solution 70% | 250.00 |
| Citric acid anhydrous | 3.47 |
| Sodium citrate dehydrate | 9.37 |
| Hydroxyethylcellulose | 1.25 |
| Poloxamer 188 | 2.0 |
| Water purified, USP | q.s. to 1 ml |

TABLE 5

| Ingredient | Amount mg/ml |
|---|---|
| 5-methyl-2-(2′-chloro-6′-fluoroanilino) | 15.0 |
| phenylacetic acid | |
| Propylparaben | 0.20 |
| Methylparaben | 1.80 |
| Simethicone emulsion 30% | 1.00 |
| Flavor | 5.0 |
| Suspending agent: Avicel ® RC591 | 12.00 |
| Propylene glycol | 25.00 |
| Sorbitol solution 70% | 250.00 |
| Citric acid anhydrous | 3.47 |
| Sodium citrate dehydrate | 9.37 |
| Sodium carboxymethylcellulose | 1.25 |
| Poloxamer 188 | 2.0 |
| Water purified, USP | q.s. to 1 ml |

TABLE 6

| Ingredient | Amount (mg/ml) |
|---|---|
| 5-methyl-2-(2′-chloro-6′-fluoroanilino) | 15.0 |
| phenylacetic acid | |
| Propylparaben | 0.16 |
| Poloxamer 188 | 2.00 |
| Methylparaben | 1.48 |
| Simethicone emulsion 30% | 2.00 |
| Flavor | 4.0 |
| Suspending agent: Avicel ® RC591 | 15.00 |
| Propylene glycol | 25.00 |
| Sorbitol solution 70% | 250.00 |
| Citric acid anhydrous | 0.71 |
| Sodium citrate dihydrate | 1.88 |
| Sodium saccharin | 0.50 |
| Water purified, USP | q.s. to 1 ml |

TABLE 7

| Ingredient | Amount (mg/ml) |
|---|---|
| 5-methyl-2-(2′-chloro-6′-fluoroanilino) | 15.0 |
| phenylacetic acid | |
| Propylparaben | 0.16 |
| Poloxamer 188 | 2.00 |
| Methylparaben | 1.48 |
| Simethicone emulsion 30% | 1.00 |
| Flavor | 4.0 |
| Suspending agent: Avicel ® RC591 | 15.00 |
| Propylene glycol | 25.00 |
| Sorbitol solution 70% | 250.00 |
| Citric acid anhydrous | 0.71 |
| Sodium citrate dihydrate | 1.88 |
| Sodium saccharin | 0.50 |
| Water purified, USP | q.s. to 1 ml |

1. A liquid oral dosage formulation comprising water, 5-methyl-2-(2′-chloro-6′-fluoroanilino)phenylacetic acid, and a suspending agent, wherein the pH of said formulation is between about 4.3 and about 5.5.

2. The liquid oral dosage formulation of claim 1, wherein said suspending agent is a member selected from the group consisting of microcrystalline cellulose, carboxymethylcellulose sodium, guar gum, xanthan gum, gellan gum, carrageenan, sodium starch glycolate, and mixtures thereof.

3. The liquid oral dosage formulation of claim 2, further comprising a wetting agent.

4. The liquid oral dosage formulation of claim 3, wherein said wetting agent is a member selected from the group consisting of polysorbate 80, poloxamers, polyethoxylated castor oil, polyethoxylated hydrogenated castor oil, polyoxyl 40 stearate, and mixtures thereof.

5. The liquid oral dosage formulation of claim 2, wherein the pH of said formulation is between about 4.5 and 5.5.

6. The liquid oral formulation of claim 5, wherein the pH of said formulation is between about 4.75 and about 5.25.

7. The liquid oral formulation of claim 4, wherein the pH of said formulation is about 5.0, and said poloxamer is poloxamer 188.

8. The liquid oral formulation of claim 1, wherein said suspending agent is a mixture of microcrystalline cellulose and carboxymethylcellulose sodium.

9. The liquid oral formulation of claim 8 comprising a buffer system.

10. The liquid oral formulation of claim 9 wherein said buffer system comprises a member selected from the group consisting of alkaline metal citrate salts with citric acid, alkaline metal acetate salts with acetic acid, alkaline metal succinate salts with succinic acid, and mixtures thereof.

11. The liquid oral formulation of claim 9, further comprising an antifoaming agent.

12. The liquid oral formulation of claim 9, further comprising a preservative.

13. The liquid oral formulation of claim 12, wherein said preservative is a member selected from the group consisting of benzoic acid, sorbic acid, butylparaben, ethylparaben, methylparaben, propylparaben, sodium benzoate, sodium propionate, and mixtures thereof.

14. A method for preparing a liquid oral suspension comprising 5-methyl-2-(2′-chloro-6′-fluoroanilino)phenylacetic acid, comprising:
admixing water, drug substance, and suspending agent, to yield a first mixture, and then admixing buffer system components; or
admixing water, suspending agent and buffer system components to yield a first mixture, and then admixing drug substance.

15. The method of claim 14, wherein said suspending agent is a mixture of microcrystalline cellulose and carboxymethylcellulose sodium.

16. The method of claim 15, wherein said liquid oral suspension has a pH of between about 4.3 and 5.5.

17. The method of claim 16, wherein said buffer system components are citric acid and sodium citrate.

18. The method of claim 17, wherein said liquid oral suspension has a pH of about 5.0.

19. A method for minimizing the dissolution and degradation of an aqueous suspension of 5-methyl-2-(2′-chloro-6′-fluoroanilino)phenylacetic acid, comprising providing an aqueous suspension of 5-methyl-2-(2′-chloro-6′-fluoroanilino)phenylacetic acid and adjusting the pH of said suspension to between about 4.3 and about 5.5.

20. The method of claim 19, wherein said pH is adjusted to about 5.0.

21. A method for treating a cyclooxygenase-2 dependent disorder or condition comprising administering an effective amount of a liquid oral dosage formulation comprising 5-methyl-2-(2′-chloro-6′-fluoroanilino)phenylacetic acid, wherein the pH of said formulation is between about 4.3 and 5.5.

22. The method of claim 22, wherein the pH of said formulation is about 5.0.
